(19) United States
(12) Patent Application Publication
(10) Pub. No.: US 2022/0208392 A1
(43) Pub. Date: Jun. 30, 2022

(54) MONITORING POTENTIAL CONTACT BASED INFECTION EVENTS

(71) Applicant: Cortica Ltd., Tel Aviv (IL)

(72) Inventor: KARINA ODINAEV, Tel Aviv, IL (US)

(73) Assignee: Cortica Ltd., Tel Aviv (IL)

(21) Appl. No.: 17/646,289

(22) Filed: Dec. 28, 2021

Related U.S. Application Data
(60) Provisional application No. 63/199,437, filed on Dec. 28, 2020.

(51) Int. Cl. G16H 50/80; G16H 50/30

(52) U.S. Cl. CPC G16H 50/80; H04W 4/029; G16H 50/30

(57) ABSTRACT

Systems, and method and computer readable media that store instructions for monitoring potential contact based infection events.

### BACKGROUND

In the recent years we have experienced many outbursts of highly infectious diseases. A single sick person may infect tens of people that may, in turn, infect many other people.

It has been found that a some infectious diseases may spread via contact of different people with the same contaminated area and/or due to ejection of infectious droplets of saliva.

There is a growing need to monitor persons involved in potential infection events.

### SUMMARY

There may be provided systems, methods, and computer readable medium as illustrated in the specification.

### BRIEF DESCRIPTION OF THE DRAWINGS

The embodiments of the disclosure will be understood and appreciated more fully from the following detailed description, taken in conjunction with the drawings in which:

FIG. 1 illustrates an example of a method;

FIG. 2 illustrates suspected persons, and a non-suspected person;

FIG. 3 illustrates an example of a method;

FIG. 4 illustrates suspected persons; and

FIG. 5 illustrates an example of a system.

### DESCRIPTION OF EXAMPLE EMBODIMENTS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the present invention may for the most part, be implemented using electronic components and circuits known to those skilled in the art, details will not be explained in any greater extent than that considered necessary as illustrated above, for the understanding and appreciation of the underlying concepts of the present invention and in order not to obfuscate or distract from the teachings of the present invention.

Any reference in the specification to a method should be applied mutatis mutandis to a device or system capable of executing the method and/or to a non-transitory computer readable medium that stores instructions for executing the method.

Any reference in the specification to a system or device should be applied mutatis mutandis to a method that may be executed by the system, and/or may be applied mutatis mutandis to non-transitory computer readable medium that stores instructions executable by the system.

Any reference in the specification to a non-transitory computer readable medium should be applied mutatis mutandis to a device or system capable of executing instructions stored in the non-transitory computer readable medium and/or may be applied mutatis mutandis to a method for executing the instructions.

Any combination of any module or unit listed in any of the figures, any part of the specification and/or any claims may be provided.

The specification and/or drawings may refer to an image. An image is an example of a media unit. Any reference to an image may be applied mutatis mutandis to a media unit. A media unit may be an example of sensed information unit. Any reference to a media unit may be applied mutatis mutandis to sensed information. The sensed information may be sensed by any type of sensors—such as a visual light camera, or a sensor that may sense infrared, radar imagery, ultrasound, electro-optics, radiography, LIDAR (light detection and ranging), etc.

The specification and/or drawings may refer to a processor. The processor may be a processing circuitry. The processing circuitry may be implemented as a central processing unit (CPU), and/or one or more other integrated circuits such as application-specific integrated circuits (ASICs), field programmable gate arrays (FPGAs), full-custom integrated circuits, etc., or a combination of such integrated circuits.

Any combination of any steps of any method illustrated in the specification and/or drawings may be provided.

Any combination of any subject matter of any of claims may be provided.

Any combinations of systems, units, components, processors, sensors, illustrated in the specification and/or drawings may be provided.

The analysis of content of a media unit may be executed by generating a signature of the media unit and by comparing the signature to reference signatures. The reference signatures may be arranged in one or more concept structures or may be arranged in any other manner. The signatures may be used for object detection or for any other use.

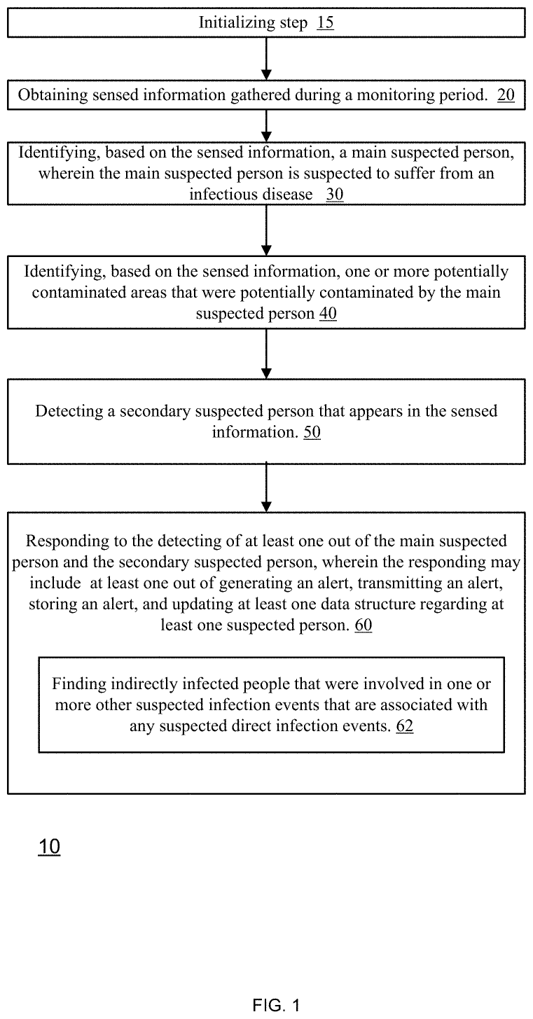

FIG. 1 illustrates method 10 for monitoring potential contact based infection events.

Method 10 may start by initializing step 15.

The initializing step 15 may include receiving infection information to be used during one or more steps of method 10.

The infection information may, for example, include definitions of what a contaminated area is and/or information regarding the chances that an area is contaminated and/or information reflecting the contamination level of an area, and/or the chances that another person will be infected when contacting the area.

The infection information may include one or more parameters that may be used (and how to use these parameters) to determine whether an area is potentially infectious and/or may be used to determine the chances that an area is contaminated and/or affect the contamination level of the area.

The infection information may include the manner in which the one or more parameters affect the infection.

The one or more parameters may include, for example, at least one of the following:
The manner in which the main suspected person contacted the area—which organ contacted the area (mouth exhibits a higher contamination risk that leg), a duration of contact, force applied, a direction of movement of the organ when contacting the area or in proximity to the contact, a contact region (for example size) between the organ of the person and the area, whether the contact was made by protected or unprotected area of the organ, and the like).
One or more actions of the main suspected person prior the contact (for example—contacting the area after sterilizing the area of the organ that contacted the area—and if so the time gap between sterilization and contact may be taken into account, contacting the area with a palm of the hand after wiping the mouth or after sneezing, and the like).
One or more main suspected person parameters—age (age statistics regarding contingency level, any health indicator obtained about the person, visible disease symptoms, and the like.
One or more environmental conditions such as rain, wind, temperature (as well as the direction of wind, wind speed, any parameter that may affect evaporation, and the like).
One or more area parameters (such as material, roughness, shape, location of the area—for example within a closed room, outdoor, and the like).
One or more non-contact parameters—such as contamination by droplets (sneezing/coughing). In this case any parameter related to method 200 may be used.
Change of the infection potential over time (following the contact). For example—determining whether the area is still contaminated and/or the risk after a time gap between the initial contact and contacts of other persons.
Affect of other contacts of the area—for example—contacts that follow the contact made by the main suspected person may remove (gradually or in any other manner) any infectious material from the area. In each of said contacts one or more of the previously mentioned parameters may be taken into account.

Step 15 may be followed by step 20 of obtaining sensed information gathered during a monitoring period. The monitoring period may be of any duration.

Step 20 may be followed by step 30.

Step 30 may include identifying, based on the sensed information, a main suspected person, wherein the main suspected person is suspected to suffer from an infectious disease. The determination may be also based on the infection information received in step 15.

The name or other identifier of the main suspected person may be provided in any—manner for example—from health records or any other database of suspected or actual sick people.

The sensed information may be processed to determine a capture of the main suspected in the sensed information.

The identifying may include applying a face recognition process on the sensed information.

Step 30 may include identifying an area as a potentially contaminated area, and optionally determining the chances that the area is actually contaminated based on one or more parameters.

Step 30 may be followed by step 40 identifying, based on the sensed information, one or more potentially contaminated areas that were potentially contaminated by the main suspected person.

Step 40 may be followed by step 50 of detecting a secondary suspected person that appears in the sensed information.

The secondary suspected person was involved in a suspected direct infection event that may include contacting one of the potentially contaminated areas under circumstances that are indicative of a potential infection by contact. The circumstances may include any one of the parameters of the infection information—including the time gap between contact by the main suspected person and the secondary suspected persons—and any event or parameter that occurred during the time gap.

Step 50 may be responsive to any of the one or more parameters or any other part of the infection information.

Step 50 may include re-evaluating the status of each potentially infectious area following each contact of a secondary suspected person—for example evaluating how much of the infectious material was removed by the contact of the secondary suspected person. The update may take into account any of the parameters in the infection information.

The re-evaluation of the status may also take into account previous events related to the secondary suspected persons—for example contacts with other potentially infectious areas—for example—a secondary suspected person may carry material from a previously contacted potentially infectious area and deposit in on an new potentially infectious area.

The method may maintain a data record per suspected infections area and update the data record with at least one out of events related to the area (contact events, whether events), the risk associated with the area (may change over time), infection potential, and the like.

Step 50 may include determining a severity of the suspected direct infection event. The determining of the severity may be executed in any manner.

Step 50 may include determining a probability of infection resulting from the suspected direct infection event. The probability may be calculated in various manners.

Step 50 may be followed by step 60 of responding to the detecting of at least one out of the main suspected person and the secondary suspected person, wherein the responding may include at least one out of generating an alert, transmitting an alert, storing an alert, and updating at least one data structure regarding at least one suspected person.

Step 60 may include step 62 of finding suspected indirectly infected people that were involved in one or more other suspected infection events that are associated with any suspected direct infection events.

Step 62 may be followed by performing any response—for example at least one out of generating an alert, transmitting an alert, storing an alert, and updating at least one data structure regarding at least one suspected person.

Method 10 may estimate over time (following contacts of an area, per time period, per event (for example rain, storm,) the state of potentially infectious areas—and update their status, calculate one or more parameters, and the like.

When first detecting any suspected person—main or secondary—his identity may be clear or not. For example—if the face of the person is images at the time of first detection—then face recognition may assist in detecting the person. If not (for example only a rear view of the person is acquired at the time of first detection)—the method may continue tracking after the person until his identity is clear—for example waiting until a detailed enough image of the face of the person is acquired.

Step 30 may include step (a) determining whether an identity of a suspected (main or secondary) infected person is clear during an occurrence of a potential direct infection event. If yes—jumping to step (c). If no—step (a) may be followed by step (b) of continuing to track after the suspected infected person until at least additional information for clarifying the identity is detected. Step (b) is followed (c) of identifying the person.

Steps (a), (b) and (c) may also be included in step 50 and/or in step 62.

Step 20 may include obtaining the sensed information from multiple sensors. For example—different cameras distributed in different locations.

Steps 30, 40 and 50 may be based on generating signatures of the sensed information.

Steps 20, 30, 40, 50 and 60 may be executed in real time (for example within less than a second, within up to a minute, up to a few minutes, and the like).

Step 60 may include generating and transmitting, in real time, alerts that notify people located at a vicinity of the main and/or secondary suspected persons, about the suspected persons.

When the method is executed in real time—there may be provided a database of sick people. When the method identified a sick person, based on the database, the method may generate an alert in real time—to notify people in the vicinity of the sick person.

Step 60 may include generating and transmitting, in real time, alerts that notify at least one access control entity (human guard, automatic gate control, and the like) of at least one asset that is located at a vicinity of at least one suspected person, about the at least one suspected person. This will allow to restrict access of persons to the asset.

Method 10 may be executed in non-real time and step 60 may include analyzing a distribution of a pandemic.

At least one of steps 30, 40 and 50 or any related identification and/or any movement analysis can be done in various manners—by applying a deep neural network, by applying a machine learning process, by generating signatures of the images, by face recognition algorithms, and the like. An example of a signature generation process and/or object detection is illustrated in U.S. patent application Ser. No. 16/542,327 now U.S. patent Ser. No. 10/748,038, which is incorporated herein by reference.

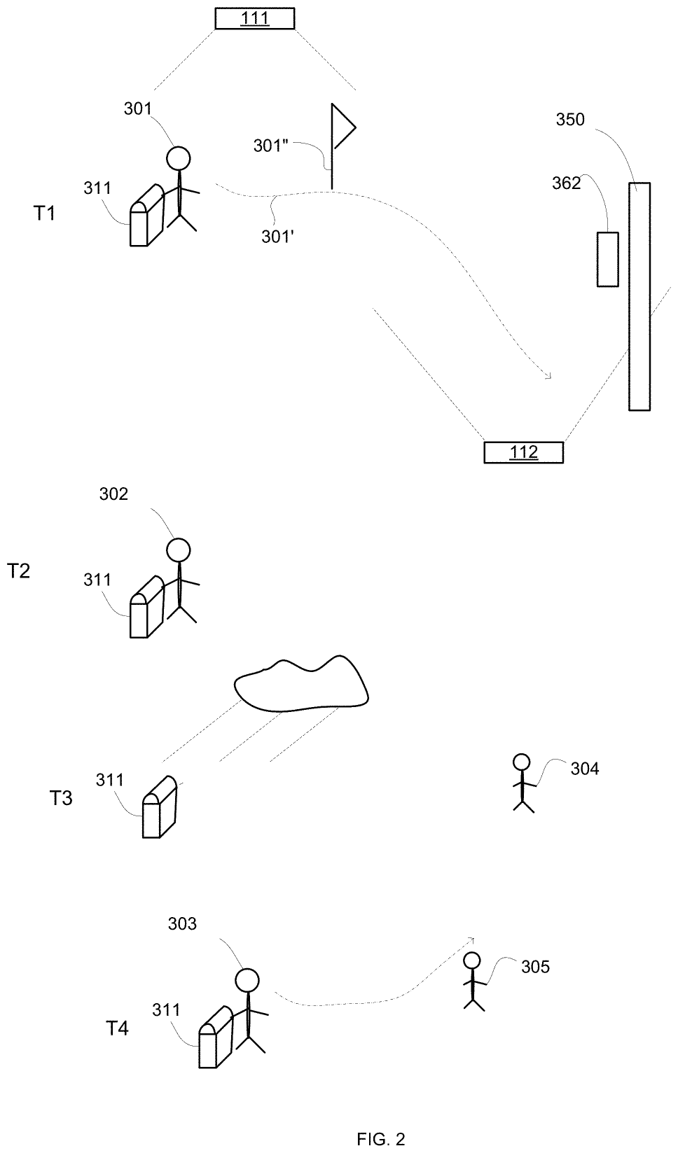

FIG. 2 illustrates a main suspected person 301, secondary suspected persons 302 and 303, suspected indirectly infected people 305 and a non-suspected person 304.

FIG. 2 illustrates four points of time—first point of time T1, second point of time T2, third point of time T3 and fourth point of time T4.

At a first point of time (T1) the main suspected person 301 (captured in sensed information from first camera 111) touches a top area of mail box 311. The identity of the main suspected person 301 is clarified at point 301″ (that may follow the time of contact).

The circumstances (parameters) of the contact are taken into account when determining whether the area is potentially contaminated—and if so—may also determine other matters such as risk potential, and the like. For simplicity of explanation it is assumed that the area is potentially contaminated.

The main suspected person 301 may follow a path (following T1) and its entrance to asset 350 may be denied by access control entity 362—following a detection of that person as a main primary suspect. Second camera 112 may view the vicinity of the asset 350.

At a second point of time (T2) another person contacts the area that is potentially contaminated. It is assumed that the other person is determined to be a secondary suspected person 302. The status of the area may be re-evaluated.

At a third point of time (T3) rain falls on mailbox 311 and is determined to remove at least some of the infectious material from mail box 311. The status of the area may be re-evaluated.

At a fourth point of time (T4) a further person contacts the area that is potentially contaminated. It is assumed that the further person is determined to be a secondary suspected person 303. The status of the area may be re-evaluated

The secondary suspected person 303 may follow (after T4) a path and may infect yet another person—suspected indirectly infected people 305.

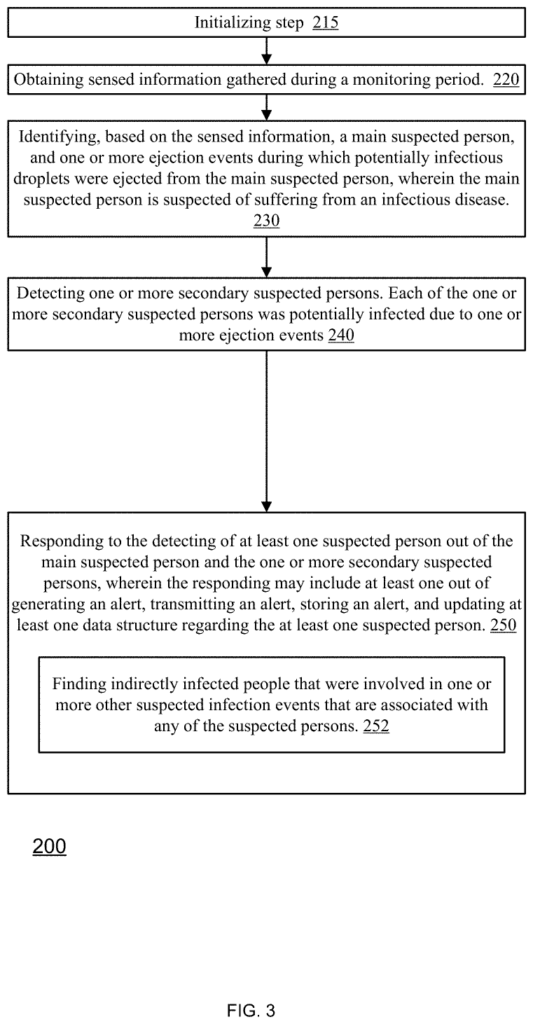

FIG. 3 illustrates method 200.

Method 200 may start by initializing step 215.

The initializing step 215 may include receiving infection information to be used during one or more steps of method 200.

The infection information may, for example, include definitions of infection parameter and how that may affect infection.

The one or more parameters may include one or more droplet transmission parameters.

Additionally or alternatively, the one or more parameters may include secondary infection parameters—that are related to one or more secondary suspected persons.

The one or more droplet transmission parameters may include an age and/or a gender of the main suspected person.

The one or more droplet transmission parameters may include a behavior of the main suspected during each of the one or more ejection events.

The behavior may be selected out of one out of sneezing, coughing, talking, any movement during any ejection event, a pose of a person during any ejection event (for example sitting or standing) attempting to block at least a part of the droplet—and the manner in which the attempts were made, estimated success or estimated failure of such attempts, and the like.

The behavior (during an ejection event) may include one out of leaving a nose and a mouth of the main suspected person unmasked, partially masking the nose and mouth of the main suspected person, and fully masking the nose and mouth of the main suspected person.

For each ejection event the infection parameters may include a spatial relationship between the main suspected person and any secondary suspected person located at the vicinity of the main suspected person. Vicinity—may be with the range of the droplets—for example up to 1, 2, 3, 4, 5, 6, 7, 8, 9, 10, 11, 12, 13, 14, 215, 16 meters and the like.

For each ejection event the infection parameters may include an amount of alignment between a mouth of the main suspected person and a mouth of any secondary suspected person located at the vicinity of the main suspected person. Additionally or alternatively the alignments may refer to the noses of the main and secondary suspected persons. Additionally or alternatively the alignments may refer to the mouth and/or nose of the main suspected person and to the mouth and/or node of any of secondary suspected persons.

The infection information may also indicate the relationship between any one or more of the infection parameters and the chances that a person can be deemed to be a secondary suspected person—or whether the secondary suspected person was infected—and/or what are the chances of infection, what is the risk of infection, the severeness of infection, and the like.

The relationships may be learnt in various manners—for example based on medical models, medical records, based on a machine learning process, and the like. Models of saliva droplet distribution may be monitored and/or simulated and/or estimated in any manner.

Step 215 may be followed by step 220 of obtaining sensed information gathered during a monitoring period.

Step 220 may be followed by step 230 of identifying, based on the sensed information, a main suspected person and one or more ejection events during which potentially infectious droplets were ejected from the main suspected person, wherein the main suspected person is suspected of suffering from an infectious disease.

Step 230 may include identifying of the main suspected person by applying a face recognition process on the sensed information.

Step 230 may be followed by step 240 of detecting one or more secondary suspected persons. Each of the one or more secondary suspected persons was potentially infected due to one or more ejection events.

The detecting is based, at least on part, on infection information—such as infection parameters and their relationship to an infection.

Step 240 may be followed by step 250 of responding to the detecting of at least one suspected person out of the main suspected person and the one or more secondary suspected persons, wherein the responding may include at least one out of generating an alert, transmitting an alert, storing an alert, and updating at least one data structure regarding the at least one suspected person.

Step 250 may include step 252 of finding indirectly infected people that were involved in one or more other suspected infection events that are associated with any of the suspected persons.

Step 252 may be followed by generating any other response—for example at least one out of generating an alert, transmitting an alert, storing an alert, and updating at least one data structure regarding the at least one suspected person.

When first detecting any suspected person—main or secondary—his identity may be clear or not. For example—if the face of the person is images at the time of first detection—then face recognition may assist in detecting the person. If not (for example only a rear view of the person is acquired at the time of first detection)—the method may continue tracking after the person until his identity is clear—for example waiting until a detailed enough image of the face of the person is acquired.

Step 230 may include step (a) determining whether an identity of a suspected (main or secondary) infected person is clear during an occurrence of a potential direct infection event. If yes—jumping to step (c). If no—step (a) may be followed by step (b) of continuing to track after the suspected infected person until at least additional information for clarifying the identity is detected. Step (b) is followed (c) of identifying the person.

Steps (a), (b) and (c) may also be included in step 240 and/or in step 252.

Step 20 may include obtaining the sensed information from multiple sensors. For example—different cameras distributed in different locations.

Steps 220, 230 and 240 may be based on generating signatures of the sensed information.

Steps 220, 230 and 240 may be executed in real time (for example within less than a second, within up to a minute, up to a few minutes, and the like).

Step 250 may include generating and transmitting, in real time, alerts that notify people located at a vicinity of the main and/or secondary suspected persons, about the suspected persons.

When the method is executed in real time—there may be provided a database of sick people. When the method identified a sick person, based on the database, the method may generate an alert in real time—to notify people in the vicinity of the sick person.

Step 250 may include generating and transmitting, in real time, alerts that notify at least one access control entity (human guard, automatic gate control, and the like) of at least one asset that is located at a vicinity of at least one suspected person, about the at least one suspected person. This will allow to restrict access of persons to the asset.

Method 200 may be executed in non-real time and step 250 may include analyzing a distribution of a pandemic.

At least one of steps 220, 230 and 240 or any related identification and/or any movement analysis can be done in various manners—by applying a deep neural network, by applying a machine learning process, by generating signatures of the images, by face recognition algorithms, and the like. An example of a signature generation process and/or object detection is illustrated in U.S. patent application Ser. No. 16/542,327 now U.S. patent Ser. No. 16/542,327 which is incorporated herein by reference.

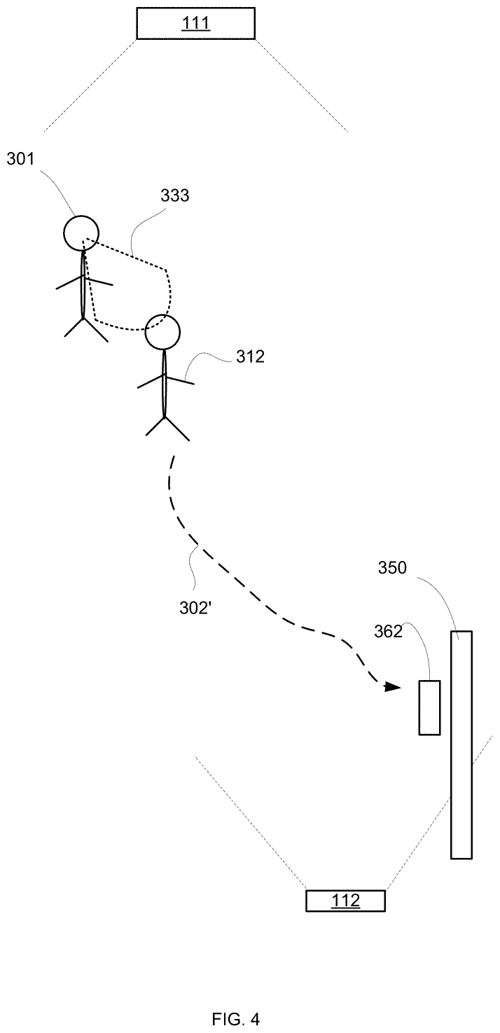

FIG. 4 illustrates a main suspected person 301, and a secondary suspected person 302.

The main suspected person 301 (captured in sensed information from first camera 111) ejects saliva droplets 333 towards another person.

The infection parameters related to this injection event are taken into account when determining whether the other person is a secondary suspected person 312. Other parameters such as the risk of infection and the like may also be evaluated.

For simplicity of explanation it is assumed that the area is potentially contaminated.

The secondary suspected person 312 may follow a path 302′ and its entrance to asset 350 may be denied by access control entity 362—following a detection of that person as secondary suspected person 312. Second camera 112 may view the vicinity of the asset 350.

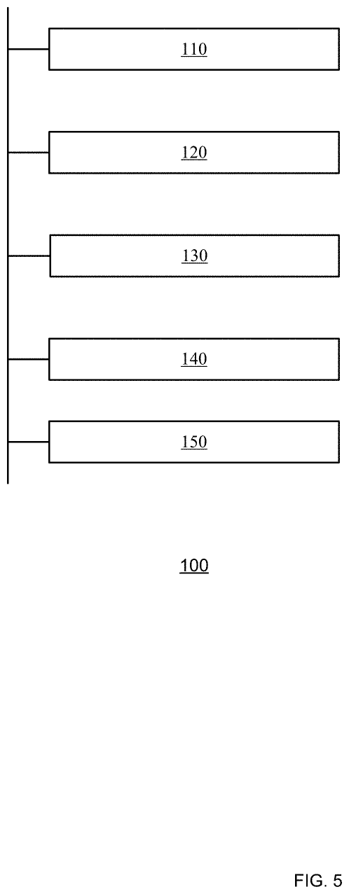

FIG. 5 illustrates a computerized system 100.

Computerized system 100 may be configured to execute one, some of all steps of method 100. Additionally or alternatively—computerized system 100 may be configured to execute one, some of all steps of method 200.

Computerized system 100 may include sensed information gathering unit 110, processor 120, response unit 130, memory unit 140 and input/output unit 150.

The processor 120 and/or the input/output unit 140 and/or memory unit 150 may perform at least some parts of the functions of the sensed information gathering unit 110.

The processor 120 and/or the input/output unit 140 and/or memory unit 150 may perform at least some parts of the response unit 130.

The sensed information gathering unit 110 is configured to gather sensed information that was sensed during a monitoring period. The sensed information gathering unit 110 may store the sensed information and/or may send the sensed information to the memory unit 140.

The sensed information gathering unit 110 may include one or more sensors. Additionally or alternatively, the sensed information gathering unit 110 may retrieve sensed information from the sensors or from any other source of sensed information (including databases).

The response unit 130 may be configured to respond to the detecting of the main suspected person and/or to any secondary suspected person. The responding may include at least one out of generating an alert, transmitting an alert, storing an alert, and updating at least one data structure regarding at least one suspected person, trigger a search for indirect suspected infected persons, and the like.

Input output unit 140 may receive and/or output information, and/or alerts and/or reports, and the like. Input output unit 140 may be any suitable communications component such as a network interface card, universal serial bus (USB) port, disk reader, modem or transceiver that may be operative to use protocols such as are known in the art to communicate either directly, or indirectly, with other elements of system 100 and/or other entities.

Processor 120 may execute at least one of steps 20, 30, 40, 50 and 60—either alone or in combination with other computerized units (of the system or outside the system).

Additionally or alternatively—Processor 120 may execute at least one of steps 220, 230, 240 and 250—either alone or in combination with other computerized units (of the system or outside the system).

Any other sequence of events may occur and any other types of determinations may be made.

It should be noted that any one of methods 10 and 200 may be applicable when multiple infection types (contact based, proximity based, ejection of droplet based) are present, and applicable to multiple suspected persons. A person may be deemed to be suspected based on one or more events of one of more types (contacting a potentially contaminated area, being within a range of potentially infectious droplets, and the like). The status of different persons and/or different potentially contaminated area may be re-evaluated overtime, and various decisions can be made and/or changed overtime.

The terms persons and people are used in an interchangeable manner.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

In the foregoing specification, the invention has been described with reference to specific examples of embodiments of the invention. It will, however, be evident that various modifications and changes may be made therein without departing from the broader spirit and scope of the invention as set forth in the appended claims.

Moreover, the terms “front,” “back,” “top,” “bottom,” “over,” “under” and the like in the description and in the claims, if any, are used for descriptive purposes and not necessarily for describing permanent relative positions. It is understood that the terms so used are interchangeable under appropriate circumstances such that the embodiments of the invention described herein are, for example, capable of operation in other orientations than those illustrated or otherwise described herein.

Furthermore, the terms “assert” or “set” and “negate” (or “deassert” or “clear”) are used herein when referring to the rendering of a signal, status bit, or similar apparatus into its logically true or logically false state, respectively. If the logically true state is a logic level one, the logically false state is a logic level zero. And if the logically true state is a logic level zero, the logically false state is a logic level one.

Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures may be implemented which achieve the same functionality.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

Also for example, in one embodiment, the illustrated examples may be implemented as circuitry located on a single integrated circuit or within a same device. Alternatively, the examples may be implemented as any number of separate integrated circuits or separate devices interconnected with each other in a suitable manner.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

It is appreciated that various features of the embodiments of the disclosure which are, for clarity, described in the contexts of separate embodiments may also be provided in combination in a single embodiment. Conversely, various features of the embodiments of the disclosure which are, for brevity, described in the context of a single embodiment may also be provided separately or in any suitable sub-combination.

It will be appreciated by persons skilled in the art that the embodiments of the disclosure are not limited by what has been particularly shown and described hereinabove. Rather the scope of the embodiments of the disclosure is defined by the appended claims and equivalents thereof

What is claimed is:

1. A method for monitoring potential contact based infection events, the method comprises:
obtaining sensed information gathered during a monitoring period;
identifying, based on the sensed information, a main suspected person, wherein the main suspected person is suspected to suffer from an infectious disease;
identifying, based on the sensed information, one or more potentially contaminated areas that were potentially contaminated by the main suspected person;
detecting a secondary suspected person that appears in the sensed information; wherein the secondary suspected person was involved in a suspected direct infection event that comprises contacting one of the potentially contaminated areas under circumstances that are indicative of a potential infection by contact; and
responding to the detecting of at least one out of the main suspected person and the secondary suspected person, wherein the responding comprises at least one out of generating an alert, transmitting an alert, storing an alert, and updating at least one data structure regarding at least one suspected person.

2. The method according to claim 1 wherein the identifying, based on the sensed information, of one of the potentially contaminated areas comprises identifying a spread of droplets that was emitted from the main suspected person on the main suspected person.

3. The method according to claim 1 wherein the identifying, based on the sensed information, of one of the potentially contaminated areas comprises determining whether a bare organ of the main suspected person contacted the potentially contaminated area.

4. The method according to claim 1 wherein the identifying, based on the sensed information, of one of the potentially contaminated area is responsive to a duration of contact of the main suspected person and the potentially contaminated area.

5. The method according to claim 1 wherein the identifying, based on the sensed information, of one of the potentially contaminated areas is responsive to an estimated force applied by the main suspected person on the potentially contaminated area.

6. The method according to claim 1 wherein the identifying, based on the sensed information, of one of the potentially contaminated areas is responsive to an estimated contact region between the main suspected person and the potentially contaminated area.

7. The method according to claim 1 wherein the identifying, based on the sensed information, of one of the potentially contaminated areas is responsive to weather conditions during a contact with the potentially contaminated area and following the contact.

8. The method according to claim 1 wherein the circumstances that are indicative of the potential infection by contact comprise a time gap between a contact by the main suspected person and a contact by the secondary suspected person.

9. The method according to claim 1 wherein the circumstances that are indicative of the potential infection by contact comprise a manner in which each one of the main suspected person and the a secondary suspected person.

10. The method according to claim 1 further comprising finding indirectly infected people that were involved in one or more other suspected infection events that are associated with any suspected direct infection events.

11. The method according to claim 1 wherein the wherein the identifying of the main suspected person comprises applying a face recognition process on the sensed information.

12. A non-transitory computer readable medium for monitoring potential contact based infection events, the non-transitory computer readable medium stores instructions for:
obtaining sensed information gathered during a monitoring period;
identifying, based on the sensed information, a main suspected person, wherein the main suspected person is suspected to suffer from an infectious disease;
identifying, based on the sensed information, one or more potentially contaminated areas that were potentially contaminated by the main suspected person;
detecting a secondary suspected person that appears in the sensed information; wherein the secondary suspected person was involved in a suspected direct infection event that comprises contacting one of the potentially contaminated areas under circumstances that are indicative of a potential infection by contact; and
responding to the detecting of at least one out of the main suspected person and the secondary suspected person, wherein the responding comprises at least one out of generating an alert, transmitting an alert, storing an alert, and updating at least one data structure regarding at least one suspected person.

13. The non-transitory computer readable medium according to claim 12 wherein the identifying, based on the sensed information, of one of the potentially contaminated areas comprises identifying a spread of droplets that was emitted from the main suspected person on the main suspected person.

14. The non-transitory computer readable medium according to claim 12 wherein the identifying, based on the sensed information, of one of the potentially contaminated areas comprises determining whether a bare organ of the main suspected person contacted the potentially contaminated area.

15. The non-transitory computer readable medium according to claim 12 wherein the identifying, based on the sensed information, of one of the potentially contaminated area is responsive to a duration of contact of the main suspected person and the potentially contaminated area.

16. The non-transitory computer readable medium according to claim 12 wherein the identifying, based on the sensed information, of one of the potentially contaminated areas is responsive to an estimated force applied by the main suspected person on the potentially contaminated area.

17. The non-transitory computer readable medium according to claim 12 wherein the identifying, based on the sensed information, of one of the potentially contaminated areas is responsive to an estimated contact region between the main suspected person and the potentially contaminated area.

18. The non-transitory computer readable medium according to claim 12 wherein the identifying, based on the sensed information, of one of the potentially contaminated areas is responsive to weather conditions during a contact with the potentially contaminated area and following the contact.

19. The non-transitory computer readable medium according to claim 12 further comprising finding indirectly infected people that were involved in one or more other suspected infection events that are associated with any suspected direct infection events.

20. The non-transitory computer readable medium according to claim 12 wherein the wherein the identifying of the main suspected person comprises applying a face recognition process on the sensed information.
